(19) United States
(12) Patent Application Publication
(10) Pub. No.: US 2026/0355496 A1
(43) Pub. Date: Nov. 12, 2026

(54) METHODS AND SYSTEMS FOR MONITORING PATIENT MOVEMENT

(71) Applicant: Abiomed, Inc., Danvers, MA (US)

(72) Inventor: Glen Fantuzzi, Danvers, MA (US)

(73) Assignee: Abiomed, Inc., Danvers, MA (US)

(21) Appl. No.: 19/649,278

(22) Filed: Apr. 16, 2026

Related U.S. Application Data
(60) Provisional application No. 63/790,421, filed on Apr. 17, 2025.

(51) Int. Cl. A61M 60/122; A61M 60/585

(52) U.S. Cl. CPC A61M 60/122; A61M 60/585; A61M 2205/04; A61M 2205/18; A61M 2205/3327; A61M 2205/502; A61M 2205/581; A61M 2205/583; A61M 2209/088; A61M 2230/63

(57) ABSTRACT

Methods and apparatus for monitoring movement of a patient associated with a mechanical circulatory support device are provided. The method includes receiving, from at least one sensor coupled to the patient, sensor data indicating movement information associated with the patient, determining whether one or more values of the sensor data is greater than a threshold value, and outputting, via a user interface associated with the mechanical circulatory support device, an alarm when the one or more values of the sensor data is determined to be greater than the threshold value.

### FIELD OF THE INVENTION

This disclosure relates to techniques for monitoring patient movement.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span one or more heart valves. The intravascular blood pump is typically disposed at the end of the catheter. Once in position, the pump may be used to assist the heart and pump blood through the circulatory system and, therefore, temporarily reduce load on the patient's heart, such as to enable the heart to recover after a heart attack. An exemplary intravascular blood pump is available from ABIOMED, Inc., Danvers, MA under the tradename Impella® heart pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. In some instances, the controller may raise alarms when operational data values fall outside predetermined values or ranges, for example if a leak, suction, and/or pump malfunction is detected. The controller may include a video display screen upon which is displayed a graphical communication interface configured to display the operational data and/or alarms.

### SUMMARY

An intravascular blood pump may be included as part of a cardiac support system. The intravascular blood pump may be inserted into the patient's vascular system at an access site and may be positioned in the heart of a patient at a particular location (e.g., across one or more valves of the heart) to ensure proper operation. After initial placement of the blood pump, movement of the patient may result in shifting of the placement of the intravascular blood pump relative to its desired placement, which may result in a loss of hemodynamic support for the patient. In some instances, movement (e.g., excessive and/or quick movements) of the patient may result in trauma (e.g., bleeding) at the access site where the blood pump enters the patient's vascular system. If not addressed by a healthcare professional, trauma at the access site may result in the patient entering a critical state. Alternatively, patients with intravascular blood pumps or other medical devices may be encouraged by their physician to walk to aid in their recovery. Tracking the movement of the patient may be challenging in the hospital environment and may allow for the physician to monitor the recovery of the patient. Some embodiments of the present disclosure relate to techniques for monitoring movements of the patient to identify patient movements that may affect proper placement of at least a portion of a cardiac support system implanted in the heart of a patient and/or may result in trauma to the patient at the access site.

In some embodiments a method is provided. The method includes receiving, from at least one sensor coupled to a patient, sensor data indicating movement information associated with the patient, determining whether one or more values of the sensor data is greater than a threshold value, and outputting an alarm on a user interface when the one or more values of the sensor data is determined to be greater than the threshold value.

In one aspect, the at least one sensor includes a first sensor coupled to a mechanical circulatory support device. In another aspect, the at least one sensor includes a sensor disposed in a wearable device configured to be worn by the patient. In another aspect, the wearable device may be an adhesive patch, a smart watch, or a fitness tracker. In another aspect, the at least one sensor includes a first sensor and a second sensor, and determining whether one or more values of the sensor data is greater than a threshold values includes determining whether the first sensor data received from the first sensor is greater than a first threshold value, and determining whether second sensor data received from the second sensor is greater than a second threshold value, the second threshold value being different from the first threshold value. In another aspect, the method further includes setting the first threshold value based on a location of the first sensor and setting the second threshold value based on a location of the second sensor. In another aspect, the method further includes setting the first threshold value based on a type of the first sensor, and setting the second threshold value based on a type of the second sensor. In another aspect, outputting an alarm when the one or more values of the sensor data is determined to be greater than the threshold value includes outputting the alarm when the first sensor data is greater than the first threshold value and/or the second sensor data is greater than the second threshold value. In another aspect, outputting the alarm when the one or more values of the sensor data is determined to be greater than the threshold value further includes outputting the alarm when the first sensor data is greater than the first threshold value and the second sensor data is greater than the second threshold value.

In another aspect, the method further includes receiving, from a second sensor coupled to an ancillary device separate from the mechanical circulatory support device, sensor data indicating movement information associated with the patient. In another aspect, the ancillary device includes a medical cart. In another aspect, the method further includes setting the threshold value based, at least in part, on location of an access site associated with the mechanical circulatory support device. In another aspect, the method further includes setting the threshold value based, at least in part, on treatment being received by the patient. In another aspect, the method further includes setting the threshold value based, at least in part, on location of the at least one sensor. In another aspect, the method further includes setting the threshold value based, at least in part, on time of day. In another aspect, the method further includes setting the threshold value based, at least in part, on a type of mechanical circulatory support device implanted in the patient.

In another aspect, the at least one sensor includes a first sensor and a second sensor, and determining whether the one or more values of the sensor data is greater than a threshold value includes weighting first sensor data received from the first sensor by a first weighting value, weighting second sensor data received from the second sensor by a second weighting value, combining the weighted first sensor data and the weighted second sensor data to determine combined sensor data, and determining whether one or more values of the combined sensor data is greater than the threshold value. In another aspect, the method further includes setting the first weighted value based, at least in part, on a location of the first sensor, and setting the second weighted value based, at least in part, on a location of the second sensor. In another aspect, the method further includes setting the first weighted value based, at least in part, on a type of the first sensor, and setting the second weighted value based, at least in part, on a type of the second senso

In another aspect, determining whether the one or more values of the sensor data is greater than the threshold value includes determining whether the one or more values of the sensor data is greater than the threshold value for a duration of time. In another aspect, the method further includes transmitting an indication of the alarm to a device separate from the mechanical circulatory support device. In another aspect, the method further includes in response to receiving an input from the device, removing the alarm from the user interface. In another aspect, the device is a wireless device. In another aspect, the device is a database configured to store a health record for the patient.

In another aspect, the method further includes transmitting an indication of the alarm to a console associated with the mechanical circulatory support device. In another aspect, the method further includes in response to receiving an input from the console, removing the alarm from the user interface. In another aspect, the user interface includes a visual display configured to display the alarm. In another aspect, the visual display is arranged on a console associated with the mechanical circulatory support device. In another aspect, the user interface includes an audio interface configured to produce an audio output based on the alarm. In another aspect, the method further includes receiving physiological information associated with the patient, determining instructions for a user to resolve the alarm based on the sensor data and/or the physiological information, and displaying the instructions to resolve the alarm on the user interface. In another aspect, the method further includes receiving physiological information associated with the patient, determining a new threshold value based, at least in part, on the physiological information, and setting the threshold value based on the new threshold value. In another aspect, the at least one sensor includes an accelerometer, a distance sensor, a velocity sensor, or a force sensor. In another aspect, the distance sensor is a global positioning sensor.

In some embodiments, a cardiac support system is provided. The cardiac support system includes a mechanical circulatory support device implanted in a patient, a console coupled to the mechanical circulatory support device, where the console is configured to display a user interface, at least one sensor coupled to the patient, and at least one computer processor. The at least one computer processor configured to receive, from the at least one sensor, sensor data indicating movement of the patient, determine whether one or more values included in the sensor data is greater than a threshold value, and output via the user interface, an alarm when the one or more values included in the sensor data is determined to be greater than the threshold value.

In another aspect, the at least one sensor coupled to the patient includes a first sensor coupled to the mechanical circulatory support device. In another aspect, the at least one sensor coupled to the patient includes a first sensor disposed in a wearable device configured to be worn by the patient. In another aspect, the wearable device includes an adhesive patch. In another aspect, the wearable device includes a smart watch. In another aspect, the wearable device includes a fitness tracker. In another aspect, the at least one sensor includes a first sensor and a second sensor, and wherein the at least one computer processor is configured to determine whether a value of the sensor data is greater than a threshold value by: determining whether first sensor data received from the first sensor is greater than a first threshold value, and determining whether second sensor data received from the second sensor is greater than a second threshold value, the second threshold value being different from the first threshold value. In another aspect, the at least one computer processor is further configured to: set the first threshold value based, at least in part, on a location of the first sensor, and set the second threshold value based, at least in part, on a location of the second sensor. In another aspect, the at least one computer processor is further configured to set the first threshold value based, at least in part, on a type of the first sensor, and set the second threshold value based, at least in part, on a type of the second sensor.

In another aspect, the at least one computer processor is configured to output an alarm when the one or more values of the sensor data is determined to be greater than the threshold value by outputting the alarm when the first sensor data is greater than the first threshold value and/or the second sensor data is greater than the second threshold value. In another aspect, the at least one computer processor is configured to output an alarm when the one or more values of the sensor data is determined to be greater than the threshold value by outputting the alarm when the first sensor data is greater than the first threshold value and the second sensor data is greater than the second threshold value. In another aspect, the at least one computer processor is further configured to receive, from a second sensor coupled to an ancillary device separate from the mechanical circulatory support device, sensor data indicating movement information associated with the patient. In another aspect, the ancillary device includes a medical cart.

In another aspect, the at least one computer processor is further configured to set the threshold value based, at least in part, on location of an access site associated with the mechanical circulatory support device. In another aspect, the at least one computer processor is further configured to set the threshold value based, at least in part, on treatment being received by the patient. In another aspect, the at least one computer processor is further configured to set the threshold value based, at least in part, on location of the at least one sensor. In another aspect, the at least one computer processor is further configured to set the threshold value based, at least in part, on time of day. In another aspect, the at least one computer processor is further configured to set the threshold value based, at least in part, on a type of mechanical circulatory support device implanted in the patient. In another aspect, the at least one sensor includes a first sensor and a second sensor, and the at least one computer processor is configured to determine whether the one or more values of the sensor data is greater than a threshold value by: weighting first sensor data received from the first sensor by a first weighting value, weighting second sensor data received from the second sensor by a second weighting value, combining the weighted first sensor data and the weighted second sensor data to determine combined sensor data, and determining whether one or more values of the combined sensor data is greater than the threshold value.

In another aspect, the at least one computer processor is further configured to set the first weighted value based, at least in part, on a location of the first sensor, and set the second weighted value based, at least in part, on a location of the second sensor. In another aspect, the at least one computer processor is further configured to set the first weighted value based, at least in part, on a type of the first sensor, and set the second weighted value based, at least in part, on a type of the second sensor. In another aspect, the at least one computer processor is configured to determine whether the one or more values of the sensor data is greater than a threshold value by determining whether the one or more values of the sensor data is greater than the threshold value for a duration of time. In another aspect, wherein the at least one computer processor is further configured to transmit an indication of the alarm to a device separate from the mechanical circulatory support device. In another aspect, the at least one computer processor is further configured to in response to receiving an input from the device, remove the alarm from the user interface. In another aspect, the device is a wireless device. In another aspect, the device is a database configured to store a health record for the patient.

In another aspect, the at least one computer processor is further configured to: in response to receiving an input from the console, removing the alarm from the user interface. In another aspect, the user interface includes a visual display configured to display the alarm. In another aspect, the user interface includes an audio interface configured to produce an audio output based on the alarm. In another aspect, the at least one computer processor is further configured to receive physiological information associated with the patient, determine instructions for a user to resolve the alarm based, at least in part, on the sensor data and/or the physiological information, and display the instructions to resolve the alarm on the user interface. In another aspect, the at least one computer processor is further configured to receive physiological information associated with the patient, determine a new threshold value based, at least in part, on the physiological information, and set the threshold value based on the new threshold value. In another aspect, the at least one sensor includes an accelerometer, a distance sensor, a velocity sensor, or a force sensor. In another aspect, the distance sensor includes a global positioning sensor.

### BRIEF DESCRIPTION OF DRAWINGS

FIG. 1 shows an illustrative heart pump device, in accordance with some embodiments.

FIG. 2 is a block diagram of a system architecture, in accordance with some embodiments.

FIG. 3 shows an illustrative patient with motion sensors, in accordance with some embodiments.

FIG. 4 is a flowchart of a process for monitoring movements of a patient having an implanted mechanical circulatory support device, in accordance with some embodiments of the present disclosure.

### DETAILED DESCRIPTION

A percutaneous mechanical circulatory support device (e.g., an intravascular blood pump) may be inserted into the heart of a patient via an access site such as the femoral artery, femoral vein, axillary artery, subclavian artery, inferior jugular vein, etc. The device may be advanced through the patient's vasculature to a particular location (e.g., across the aortic valve, pulmonary valve, or another location) to provide hemodynamic support to the patient. A controller associated with the mechanical circulatory support device may be configured to control one or more operations of the device and/or receive data from the device. The received data may be used to display one or more indications of the status of the mechanical circulatory support device on a user interface associated with the controller to enable physicians or other healthcare providers to monitor the operation of the mechanical circulatory support device implanted in the patient.

Some conventional controllers for mechanical circulatory support devices are not configured to monitor movement of the patient, which if excessive, may result in trauma due to movement of the implanted device. Some embodiments of the present disclosure relate to techniques for detecting movement of a patient using one or more sensors (e.g., an accelerometer, a velocity sensor, etc.) associated with a mechanical circulatory support device. Signal(s) sensed by the one or more sensors may be analyzed to identify patient movements that may result in movement of the mechanical circulatory support device sufficient to cause trauma as a result of the device movement. When such movement is detected, the controller may be configured to output an alarm to alert a user that user intervention may be needed. In some embodiments, the signal(s) sensed by the one or more sensors may be analyzed to identify patient movements to track recovery of the patient based on an amount of walking (e.g., ambulation) done by the patient. When such walking is detected, the controller may be configured to update the patient's electronic health records for the physician to review.

To provide an overall understanding of the systems, methods, and devices described herein, certain illustrative examples will be described. Although various examples may describe specific medical procedures and/or uses of a cardiac support device, it will be understood that the technology described herein may be employed in any suitable context.

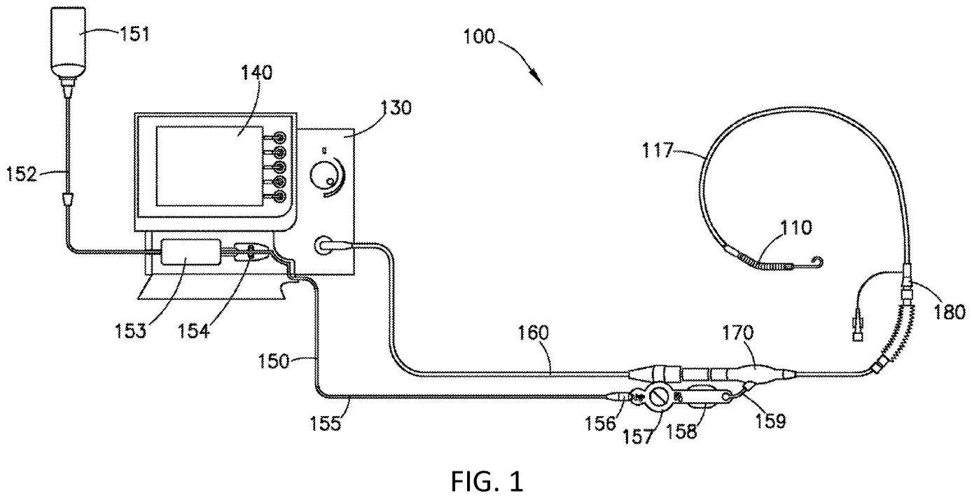

A circulatory support device (also referred to herein as a “heart pump” or simply a “pump”) may include a percutaneous, catheter-based device that provides hemodynamic support to the heart of a patient. As shown in FIG. 1, heart pump 110 may form part of a cardiac support system 100. Cardiac support system 100 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of heart pump 110. During operation, purge subsystem 150 may be configured to deliver a purge fluid to heart pump 110 through catheter tube 117 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between heart pump 110 and controller 130. Plug 170 may connect catheter tube 117, purge subsystem 150, and connector cable 160. In some implementations, plug 170 may include a storage device (e.g., a memory) configured to store, for example, operating parameters to facilitate transfer of the patient to another controller if needed. Repositioning unit 180 may be used to reposition heart pump 110 in the patient's heart.

As shown in FIG. 1, in some embodiments, the cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of heart pump 110 and purge disc 154. Controller 130 may also be configured to control operation of the motor (not shown) of the heart pump 110 and purge cassette 153. As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the heart pump 110 (e.g., one or more pressure sensors) and controller 130. Motor cables (not shown) within catheter tube 117, connector cable 160, and plug 170 may provide an electrical connection between the motor of the heart pump 110 and controller 130. During operation, controller 130 may be configured to receive measurements from one or more pressure sensors of the heart pump 110 through the sensor cables (e.g., optical fibers) and to control the electrical power delivered to the motor of the heart pump 110 through the motor cables. By controlling the power delivered to the motor of the heart pump 110, controller 130 may be operable to control the speed of the motor.

Various modifications can be made to cardiac support system 100 and one or more of its components. For instance, one or more additional sensors may be added to heart pump 100. In another example, a signal generator may be added to heart pump 100 to generate a signal indicative of the rotational speed of the motor of the heart pump 110. As another example, one or more components of cardiac support system 100 may be separated. For instance, display 140 may be incorporated into another device in communication with controller 130 (e.g., wirelessly or through one or more electrical cables).

A heart pump (e.g., heart pump 110) may include a pressure sensor (e.g., an optical pressure sensor) configured to detect a pressure within the aorta of a patient's heart when the heart pump is properly positioned in the left side of the heart. The pressure signal sensed by the pressure sensor may be used, at least in part, to determine correct positioning of the heart pump within the patient's heart and/or to determine a blood flow rate through the heart pump when in operation. For instance, the pressure signal may be used in combination with a motor current signal received from a motor current sensor (not shown) and a set of stored values to determine a flow rate of blood through the heart pump. The differential pressure across the aortic valve may also indirectly be determined based on the pressure signal measuring the pressure in the aorta and the set of stored values.

As described herein, proper placement of a heart pump within a patient's heart may be important for safety and efficacy of the device during operation. For instance, migration of the pump away from its proper placement (e.g., across the aortic valve for a device placed in the left side of the heart) may result in one or more of reduced pump flow and compromised efficacy, increased hemolysis, potential internal bleeding and poor outcomes for the patient, a poor user experience, premature removal of the pump, and/or reduced future utilization of the pump. Additionally, some (e.g., excessive) patient movement may result in trauma at the access site where the pump is inserted into the patient's vascular system. The inventors have recognized and appreciated that patient safety may be improved by monitoring the movement of patients with implanted heart pumps to be able to proactively alert healthcare providers when excessive patient movements (e.g., movements beyond a threshold amount of movement) are detected.

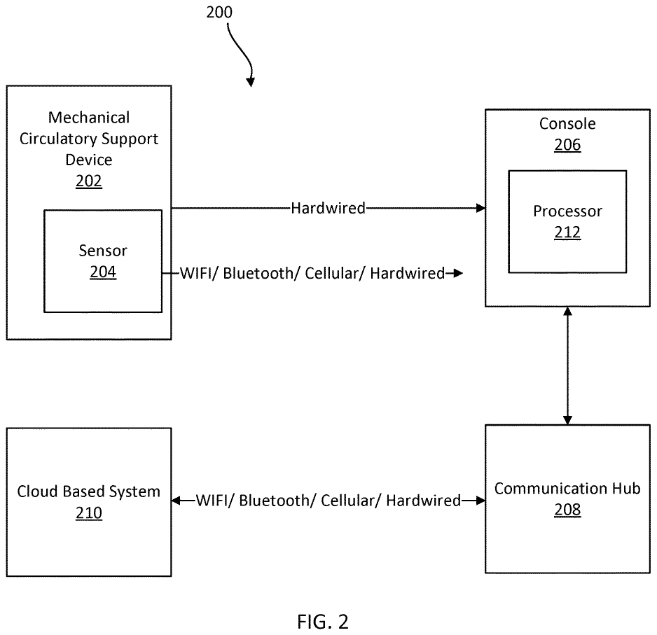

FIG. 2 schematically illustrates an example system 200 in accordance with some embodiments of the present disclosure. The system 200 may include a mechanical circulatory support (MCS) device 202 such as, for example, the heart pump 110 as illustrated in FIG. 1. The MCS device 202 may be inserted in the heart of a patient. In some embodiments, the MCS device 202 may include one or more sensors 204 configured to sense patient movement. The one or more sensors 204 may be located (e.g., be coupled to), for example, one or more portions of the MCS device 202. For instance, sensor 204 may be coupled to the plug 170 of the MCS device 202, which may be located at or near an access site where the catheter tube 117 of the MCS device 202 enters the patient's body. In some embodiments, sensor 204 may include multiple sensors coupled to the MCS device 202 at various locations. For example, an MCS device 202 may include multiple sensors coupled to any one of or a combination of, the purge subsystem 150, the display 140, the heart pump 110, the plug 170, the catheter tube 117, or any other suitable portion of MCS device 202. In some embodiments, in addition to or alternatively to sensor(s) coupled to the MCS device 202, sensor 204 may be arranged external to the MCS device 202. For instance, one or more sensors 204 may be coupled to and/or included within ancillary equipment (e.g., a medical cart) and/or one or more patient wearable devices (e.g., a smart watch, a fitness tracker, an adhesive patch).

In some embodiments, sensor data sensed by sensor 204 may indicate an amount and/or type of movement of the patient to which sensor 204 is coupled. The sensor data may, for example, sense an acceleration, a velocity, a force, any combination thereof, or any other suitable quantity associated with movement of the patient.

In some embodiments, the system 200 may include a console 206 communicatively coupled to the MCS device 202. For example, console 206 may be hardwired (e.g., using one or more cables) to the MCS device 202 to enable the transfer of information and/or operating conditions between console 206 and the MCS device 202. In some embodiments, sensor 204 may be coupled to console 206. For example, sensor 204 may be communicatively coupled to console 206 via a wireless communication protocol (e.g., WiFi, Bluetooth, cellular, etc.), and/or via a wired connection (e.g., using one or more cables) In some embodiments, console 206 may be mechanically coupled to a medical cart or other medical apparatus that enables console 206 to be maneuvered and/or positioned for use by a healthcare provider.

In some embodiments, console 206 may include a display and/or a communication interface configured to display, or otherwise provide, patient health information and/or performance metrics associated with the MCS device 202 to a user. In some embodiments, console 206 may be coupled to a cloud based system 210 via a communication hub 208. In some embodiments, console 206 may be configured to transmit patient health information (e.g., patient physiological information) to cloud based system 210. Cloud based system 210 may include, for example, one or more computing devices configured to store electronic health records and/or information associated with other patient support devices. In some embodiments, cloud based system 210 may be configured to transmit information included in a patient's electronic health records to console 206.

In some embodiments, system 200 may include a processor 212 configured to process sensor data received from sensor 204 to, for example, determine movement information associated with the patient. In various implementations, processor 212 may be located in any suitable component of system 200. For example, processor 212 may be included in console 206 and/or in cloud based system 210. In some embodiments, processor 212 may be configured to compare one or more values included in the sensor data with a threshold value. The threshold value may represent, for example, an amount of movement that may result in movement of the MCS device 202 that should be investigated by a healthcare provider. When it is determined that the one or more values included in the sensor data exceed the threshold value, an alarm may be provided to the user (e.g., on a user interface displayed by console 206) to alert the user to the excessive patient movement. In some embodiments, the alarm may be output when the sensor data from sensor 204 located at a single location exceeds its threshold value, whereas in other embodiments, the alarm may be output only when sensor data received from sensors 204 at multiple locations exceeds the threshold value.

Upon receiving the alarm, the healthcare provider may examine the access site of the MCS device and/or evaluate the placement of the MCS device 202 within the heart of the patient (e.g., using imaging) to determine whether action should be taken to reposition the device. In some embodiments, the alarm may be output via audio, visual, vibratory, or any combination thereof. In some embodiments, the alarm may be output when the value(s) of the sensor data exceeds the threshold value for a certain amount of time (e.g., to reduce false positives due to transient movements by the patient). The certain amount of time may be based, at least in part, on one or more parameters including, but not limited to, the time of day, a placement of the sensor, patient demographic information, a type of surgery the patient is recovering from, or any other suitable parameter.

In some embodiments, processor 212 may be configured to determine the threshold value and/or update the threshold value based, at least in part, on one or more parameters including, but not limited to, a placement of the sensor, a time of day, an access site location, a type of MCS device implanted in the patient, a treatment and/or medication being received by the patient (e.g., blood thinning medication), clinical indicators associated with the physiological information of the patient, or any combination thereof. For example, in some embodiments, processor 212 may be configured to use a lower threshold value when the sensor 204 is located close to the access site of the MCS device 202 as compared to when the sensor 204 is located farther away from the access site (e.g., when the sensor 204 is coupled to the plug 170). In another example, processor 212 may be configured to use a lower threshold value at a first time of day (e.g., at night) when the patient is more likely to be sleeping and/or the medical facility (e.g., a hospital) where the patient is located may have less staff for monitoring patients, as compared to a threshold value used during a second time of day (e.g., during daylight hours) when the patient is more likely to be awake and/or the medical facility may have more staff present. In some embodiments, the threshold value may be set based on one or more patient characteristics. For example, a patient with an access site more prone to bleeding may use a lower threshold than a patient with an access site that is less prone to bleeding. In some embodiments, processor 212 may be configured to use a patient's medical information, such as information about the patient's recovery plan and/or associated medication for the patient to determine the threshold value. For example, the patient's medical information may specify that a patient is receiving anticoagulant therapy, which may present a higher risk of bleeding. Based on this information, the processor 212 may be configured to use a lower threshold value. In some embodiments, processor 212 may be configured to use physiological information, such as blood tests, and clinical indicators to determine the threshold value. The blood tests may include a Heparin Anti-Xa (AntiXa) test, a partial thromboplastin time (PTT) test, an activated clotting time (ACT) test and/or a hemoglobin blood test. The AntiXa, PTT, and ACT tests may be used to indicate the anticoagulation status of the patient's blood. For example, the patient's hemoglobin blood test may indicate that the patient's blood is plasma free indicating hemolysis. Based on this information, the processor 212 may be configured to use a lower threshold value. In another example, the patient's hemoglobin count may indicate that the patient is bleeding. Based on this information, the processor 212 may be configured to use a lower threshold value and/or output the alarm.

In some embodiments, processor 212 may be configured to update the threshold value based, at least in part, on one or more inputs provided by a healthcare provider and/or a recovery status of the patient. For example, the threshold value may be updated when the patient receives a new medication that affects properties of the blood that change the bleeding risk of the patient.

In some embodiments, an indication of the alarm may provided on the console 206. Additionally or alternatively, an indication of the alarm may be transmitted from processor 212 to a device associated with the MCS device 202 (e.g., a remote monitor) and/or the cloud based system 210 configured to store a health record for the patient. In some embodiments, processor 212 may be configured to transmit an indication of the alarm to a wireless device (e.g., a smartphone, a tablet computer, etc.) associated with a nurse or other healthcare provider to inform the healthcare provider that the patient has exhibited movement, which may have adversely affected the placement of the MCS device 202 in the patient. In some embodiments, the alarm may be stored by the cloud based system 210, which may enable tracking of the amount of movement of the patient over time.

In some embodiments, processor 212 may be configured to output one or more instructions describing next steps that a healthcare provider may take to resolve the alarm. For example, processor 212 may be configured to output instructions advising the healthcare provider to perform imaging of the access site to identify potential bleeding at the access site due to the patient movement that resulted in the alarm being triggered.

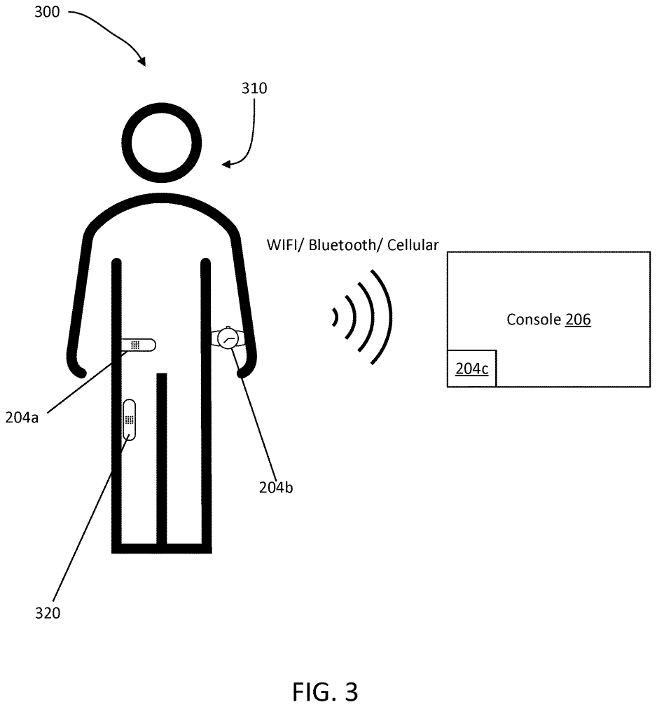

FIG. 3 illustrates an example system 300 in accordance with some embodiments of the present disclosure. System 300 illustrates sensor 204 being located at different places associated with a patient 310. Although three different locations for sensor 204 are shown in FIG. 3, it should be appreciated that such locations are provided merely as examples, and sensor 204 may be placed in any other suitable location. The patient 310 may have an access site 320 (e.g., the femoral artery) where a MCS device (e.g., MCS device 202) is inserted into the vascular system of the patient. In some embodiments, sensor 204 may be located near access site 320 to detect movement of the patient that may result in movement of the MCS device, and possible resultant bleeding at the access site 320. For instance, as shown in FIG. 3, sensor 204 may be integrated into a wearable sensor 204a (e.g., a patch such as an adhesive bandage) affixed to the patient 310 near access site 320 to sense movement of the patient at or near that location. It should be appreciated, however, that movement of the patient at or near the access site 320 may alternatively be determined by one or more sensors 204 located remote from access site 320. For example, sensor 204b may be integrated into a wearable device (e.g., smart watch, fitness tracker, necklace, ring, etc.) configured to be worn by the patient 310.

In some embodiments, sensor 204 may be coupled to medical equipment separate from but associated with an MCS device implanted in a patient. For instance, sensor 204c may be coupled to a wheeled medical cart configured to support the console 206. Sensor 204c may sense movement information for the patient 310 as the medical cart and coupled console are moved as the patient ambulates during recovery.

It should be appreciated that the location of sensor 204 and the number of sensors used may be based, at least in part, on one or more parameters including but not limited to healthcare provider experience, healthcare provider preference, type of MCS device, patient preference, patient recovery plan, other suitable parameters or any combination thereof.

In some embodiments, a threshold value used to determine whether an alarm should be triggered based on sensor data received from sensor 204 may be selected based on the location and/or type of the sensor. For example, a processor (e.g., processor 212) configured to receive sensor data may be further configured to determine a location and/or type of the sensor from which the sensor data was received. The determination may be made in any suitable way. For example, the processor may be configured to determine the location and/or type of sensor based, at least in part, on the type of sensor data and/or a unique identifier included in the sensor data. After determining the location and/or type of sensor associated with the sensor data, the processor may be configured to set an appropriate threshold value for the sensor (e.g., using stored values associated with the sensor type and/or location). For example, as shown in FIG. 3, the processor may receive sensor data from sensor 204a and sensor 204c and determine a different threshold value for each of sensor 204a and 204c based, at least in part, on the location and/or type of the respective sensor.

In some embodiments, the sensor data from sensors located at multiple locations may be used to determine whether an alarm should be triggered. In such embodiments, the sensor data may be weighted differently based, at least in part, on the location of the sensor associated with the sensor data. For example, as shown in FIG. 3, sensor data received from sensor 204a may be given greater weight than sensor data received from sensor 204c.

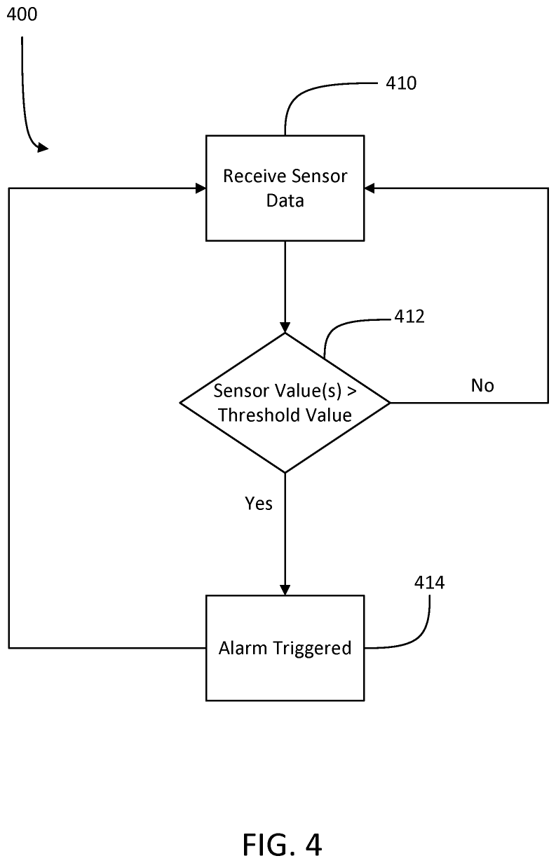

FIG. 4 illustrates a process 400 for monitoring movement information associated with a patient having an implanted MCS device (e.g., MCS device 202), in accordance with some embodiments of the disclosure. Process 400 may start in act 410, where sensor data indicating movement of the patient is received by a processor (e.g., processor 212). For example, as described herein, sensor data may be received from at least one sensor (e.g., sensor 204) coupled to the patient, MCS device, ancillary medical equipment (e.g., a medical cart) associated with the patient, or any combination thereof.

Process 400 may then proceed to act 412, where it may be determined whether one or more values included in the sensor data are greater than a threshold value. The threshold value(s) may be determined using any suitable parameter, example of which are described herein. In some embodiments, patient information may be received from a database (e.g., an electronic health record database) configured to store a health record for the patient. The patient information may include a reason for which the patient has the implanted MCS device, patient demographic information, a patient recovery plan, patient medication and/or any other information related to the patient. In some embodiments, the threshold value may be updated based, at least in part, on healthcare provider input, physiological information, sensor data, the recovery status of the patient, patient information, or any combination thereof. In some embodiments in which sensor data is received from multiple sensors, the sensor data from one or more sensors may be compared with an associated threshold value based, at least in part, on the location of the multiple sensors. Additionally or alternatively, at least some of the sensor data may be combined in some way (e.g., added, multiplied, etc.) and one or more values of the combined sensor data may be compared to a threshold value to determine whether the movement of the patient warrants generating an alarm.

If it is determined that the one or more sensor values are not greater than the threshold value, process 400 may return to act 410 where sensor data indicating movement of the patient is received until the one or more sensor values are greater than the threshold value.

When it is determined that the one or more sensor values are greater than the threshold value, process 400 may then proceed to act 414, where an alarm may be output. In some embodiments, the alarm may be displayed on a user interface associated with the MCS device. In some embodiments, outputting the alarm may include transmitting an indication of the alarm to a wireless device associated with the MCS device, the console associated with the patient, a database configured to store a health record for the patient, or any combination thereof. In some embodiments, in response to receiving an input from the wireless device or the console, the alarm may be removed from the communication interface. In some embodiments, outputting an alarm may include outputting instructions for a healthcare provider to resolve the alarm. For instance, the instructions may be displayed on a user interface associated with the console to advise the healthcare provider on potential actions for resolving the alarm. In some embodiments, the alarm may be provided audibly, using tactile feedback, or using a combination of auditory, tactile and/or visual feedback. It should be appreciated that process 400 may be performed continuously and/or at any suitable interval. In some embodiments, a processor associated with the heart pump may be configured to output an alarm only when it is determined that the sensor value(s) exceed the threshold value for a certain amount of time. For instance, the alarm may become more visible or louder if the sensor values have exceeded the threshold values for an extended period of time or if the alarm has been triggered for longer than a set period of time. The alarm may become more visible and/or louder to alert the healthcare provider of the severity of the movement of the patient.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of monitoring movement of a patient associated with a mechanical circulatory support device, the method comprising:
receiving, from at least one sensor coupled to the patient, sensor data indicating movement information associated with the patient;
determining, using at least one processor, whether one or more values of the sensor data is greater than a threshold value; and
outputting, via a user interface associated with the mechanical circulatory support device, an alarm when the one or more values of the sensor data is determined to be greater than the threshold value.

2. The method of claim 1, wherein the at least one sensor coupled to the patient includes a first sensor coupled to the mechanical circulatory support device.

3. The method of claim 1, wherein the at least one sensor coupled to the patient includes a first sensor disposed in a wearable device configured to be worn by the patient.

4. The method of claim 3, wherein the wearable device comprises an adhesive patch, a smart watch, or a fitness tracker.

5. (canceled)

6. (canceled)

7. The method of claim 1, wherein the at least one sensor includes a first sensor and a second sensor, and wherein determining whether one or more values of the sensor data is greater than a threshold value comprises:
determining whether first sensor data received from the first sensor is greater than a first threshold value; and
determining whether second sensor data received from the second sensor is greater than a second threshold value, the second threshold value being different from the first threshold value.

8. The method of claim 7, further comprising:
setting the first threshold value based, at least in part, on a location of the first sensor and/or a type of the first sensor; and
setting the second threshold value based, at least in part, on a location of the second sensor and/or a type of the second sensor.

9. (canceled)

10. The method of claim 7, wherein outputting an alarm when the one or more values of the sensor data is determined to be greater than the threshold value comprises outputting the alarm when the first sensor data is greater than the first threshold value and/or the second sensor data is greater than the second threshold value.

11. (canceled)

12. The method of claim 1, further comprising:
receiving, from a second sensor coupled to an ancillary device separate from the mechanical circulatory support device, sensor data indicating movement information associated with the patient.

13. The method of claim 12, wherein the ancillary device comprises a medical cart.

14. The method of claim 1, further comprising:
setting the threshold value based, at least in part, on one or more of a location of an access site associated with the mechanical circulatory support device, a treatment being received by the patient, a location of the at least one sensor, a time of day, or a type of mechanical circulatory support device implanted in the patient.

15-18. (canceled)

19. The method of claim 1, wherein the at least one sensor includes a first sensor and a second sensor, and wherein determining whether the one or more values of the sensor data is greater than a threshold value comprises:
weighting first sensor data received from the first sensor by a first weighting value;
weighting second sensor data received from the second sensor by a second weighting value;
combining the weighted first sensor data and the weighted second sensor data to determine combined sensor data; and
determining whether one or more values of the combined sensor data is greater than the threshold value.

20. The method of claim 19, further comprising:
setting the first weighting value based, at least in part, on a location of the first sensor and/or a type of the first sensor; and
setting the second weighting value based, at least in part, on a location of the second sensor and/or a type of the second sensor.

21. (canceled)

22. The method of claim 1, wherein determining whether the one or more values of the sensor data is greater than the threshold value comprises determining whether the one or more values of the sensor data is greater than the threshold value for a duration of time.

23. The method of claim 1, further comprising:
transmitting an indication of the alarm to a device separate from the mechanical circulatory support device; and
in response to receiving an input from the device, removing the alarm from the user interface.

24 6. (canceled)

27. The method of claim 1, further comprising:
transmitting an indication of the alarm to a console associated with the mechanical circulatory support device; and
in response to receiving an input from the console, removing the alarm from the user interface.

28. (canceled)

29. The method of claim 1, wherein the user interface comprises a visual display configured to display the alarm and/or an audio interface configured to produce an audio output based on the alarm.

30. (canceled)

31. (canceled)

32. The method of claim 1, further comprising:
receiving physiological information associated with the patient;
determining instructions for a user to resolve the alarm based, at least in part, on the sensor data and/or the physiological information; and
displaying the instructions to resolve the alarm on the user interface.

33. The method of claim 1, further comprising:
receiving physiological information associated with the patient;
determining a new threshold value based, at least in part, on the physiological information; and
setting the threshold value based on the new threshold value.

34. The method of claim 1, wherein the at least one sensor comprises one or more of an accelerometer, a distance sensor, a velocity sensor or a force sensor.

35-38. (canceled)

39. A cardiac support system comprising:
a mechanical circulatory support device implanted in a patient;
a console coupled to the mechanical circulatory support device, wherein the console is configured to display a user interface;
at least one sensor coupled to the patient; and
at least one computer processor configured to:
receive, from the at least one sensor, sensor data indicating movement of the patient;
determine whether one or more values included in the sensor data is greater than a threshold value; and
output via the user interface, an alarm when the one or more values included in the sensor data is determined to be greater than the threshold value.

40-74. (canceled)
